(19) United States
(12) Patent Application Publication
(10) Pub. No.: US 2023/0052865 A1
(43) Pub. Date: Feb. 16, 2023

(54) MOLECULAR GRAPH REPRESENTATION LEARNING METHOD BASED ON CONTRASTIVE LEARNING

(71) Applicant: ZHEJIANG UNIVERSITY, HANGZHOU, Zhejiang Province (CN)

(72) Inventors: HUAJUN CHEN, HANGZHOU, ZHEJIANG PROVINCE (CN); YIN FANG, HANGZHOU, ZHEJIANG PROVINCE (CN); HAIHONG YANG, HANGZHOU, ZHEJIANG PROVINCE (CN); XIANG ZHUANG, HANGZHOU, ZHEJIANG PROVINCE (CN); ZHUO CHEN, HANGZHOU, ZHEJIANG PROVINCE (CN)

(21) Appl. No.: 17/792,167

(22) PCT Filed: Dec. 3, 2021
(86) PCT No.: PCT/CN2021/135524

§ 371 Date: Jul. 12, 2022

(30) Foreign Application Priority Data
Dec. 25, 2020 (CN) 202011564310.8

(51) Int. Cl. G06N 3/08

(52) U.S. Cl. CPC G06N 3/08

(57) ABSTRACT

The present invention is a molecular graph representation learning method based on contrastive learning, the method comprising: obtaining a molecular fingerprint representation of each molecule, and calculating a similarity between each two molecular fingerprints; collecting a full amount of chemical functional group information, and matching a corresponding functional group for each atom in the molecule; using a heterogeneous graph to model a molecular graph; using a RGCN in the structure-aware molecular encoder to encode the representation of each atom in the molecule and the representation of the functional group to which the atom belongs, and mapping the molecule to a feature space through an aggregation function to obtain a structure-aware feature representation; according to the fingerprint similarity between molecules, selecting positive and negative samples, and carrying out a comparative learning in the feature space; obtaining the structure-aware molecular encoder by using the contrastive learning method for training on a large-sample molecular dataset, and applying the structure-aware molecular encoder to a prediction task of downstream molecular attributes. The present invention helps to capture more abundant molecular structure information and solve the problem on molecular property prediction.

### FIELD OF TECHNOLOGY

The present invention belongs to the field of graph representation learning, and in particular to a molecular graph representation learning method based on contrastive learning.

### BACKGROUND TECHNOLOGY

Over the past few years, graph representation learning has become a hot research area for analyzing graph structure data. Graph representation learning aims to learn an encoding function that takes full advantage of graph data to transform the graph data with complex structures into dense representations in a low-dimensional space that preserve diverse graph attributes and structural features.

A traditional unsupervised graph representation learning method transforms a graph into a sequence of nodes using a random walk method, and models a co-occurrence relationship between the central node and each neighbor nodes. However, such learning framework has two obvious shortcomings: one is the lack of parameter sharing between encoders, which will take up too much computing resources; the other is the lack of generalization ability of the model, which is difficult to apply to new graphs.

In recent years, graph representation learning using a Graph Neural Network (GNN) has received extensive attention. The graph neural network usually updates a hidden state of a node by a weighted sum of neighborhood states. By passing information between nodes, the graph neural network is able to capture information from its neighborhood.

Molecular graphs are a type of natural graph data with rich structural information. At present, many studies have used deep learning methods to encode molecules to accelerate drug development and molecular identification. To represent the molecules in a vector space, traditional molecular fingerprints attempt to encode the molecules as fixed-length binary vectors, with each bit on the molecular fingerprint corresponding to a molecular fragment.

In order to improve the expression ability of the molecular fingerprints, some studies have introduced the graph neural network, which takes Simplified Molecular Input Line Entry System (SMILES) representations of the molecules as an input, learns the representations of the molecules in a low-dimensional vector space, and applies them to downstream tasks such as a property prediction.

However, an experimental process of obtaining molecular property labels is time-consuming and resource-consuming, and molecular tasks face problems such as insufficient labeling data. Meanwhile, due to the extremely large molecular space, the generalization ability of the model is generally poor. To improve the generalization ability of the neural networks, some work tries to build pre-trained models on the graph representations of the molecules. Most of the work uses the types of atoms as labels in pre-trained node-level tasks, but since there are few types of atoms in the molecules, and there are cases where individual atoms frequently appear in almost all the molecules, the trained model may not be able to capture valuable chemical field information. However, in supervised graph-level tasks, models obtained by label training often only involve some specific knowledge, and most molecules lack labeling, which also limits the use of the model in practical scenarios.

Therefore, there is an urgent need to design a new molecular graph representation learning method to solve the above problems existing in the prior art.

### SUMMARY OF THE INVENTION

The present invention provides a molecular graph representation learning method based on contrastive learning, which can obtain a molecular graph representation with field information and distinctiveness, and solve the problems such as a molecular attribute prediction.

A molecular graph representation learning method based on contrastive learning, wherein, the method comprises the following steps:

(1) obtaining a molecular fingerprint representation of each molecule, and calculating a similarity between each two molecular fingerprints;

(2) collecting a full amount of chemical functional group information, and matching a corresponding functional group for each atom in the molecule; wherein, when an atom belongs to a plurality of functional groups, a functional group containing a larger number of atoms is preferentially matched;

(3) using a heterogeneous graph to model a molecular graph, wherein the heterogeneous graph is a graph containing different types of nodes and edges, different atoms correspond to different node types, and different bonds correspond to different edge types;

(4) constructing a structure-aware molecular encoder, using a relational graph convolutional network (RGCN) in the structure-aware molecular encoder to encode the representation of each atom in the molecule and the representation of the functional group to which the atom belongs, and mapping the molecule to a feature space through an aggregation function to obtain a structure-aware feature representation;

(5) according to the fingerprint similarity between molecules, selecting positive and negative samples, and carrying out a comparative learning in the feature space;

(6) obtaining the structure-aware molecular encoder by using the contrastive learning method for training on a large-sample molecular dataset, and applying the structure-aware molecular encoder to a prediction task of downstream molecular attributes.

The present invention uses the similarities of the molecular fingerprints as the basis for selecting the positive and negative samples, compares them with molecular data in the feature space, and integrates chemical field knowledge into the molecular representations, so as to obtain the molecular graph representation with field information and distinctiveness, to solve problems such as the molecular property prediction.

In step (1), the SMILES representation of the molecules is transformed into the molecular fingerprint by Rdkit which is a powerful tool for cheminformatics. According to different calculation methods, different kinds of molecular fingerprints of the same molecule can be obtained.

The molecular fingerprint is selected as one of Morgan fingerprint, Molecular ACCess System (MACCs) fingerprint and topological fingerprint. The Morgan fingerprint sets a radius from a specific atom to count the number of partial molecular structures within the radius to form the molecular fingerprint. The MACCs fingerprint pre-specifies the partial molecular structures of 166 molecules, and when the molecular structure is contained, the corresponding position is recorded as 1, otherwise it is recorded as 0. The topological fingerprint does not need to pre-specify the partial molecular structures, but calculates all molecular paths between the minimum number of bonds and the maximum number of bonds, and hashes each subgraph to generate an ID of each bit, and then generates the molecular fingerprint.

The evaluation method often used in the calculation of similarity between compound molecules is a Tanimoto coefficient. The similarity between two molecular fingerprints is calculated using the Tanimoto coefficient, and the formula is as follows:

$${S}_{AB} = \frac{c}{a + b - c}$$

wherein, a and b respectively represent the number of 1 displayed in the A and B molecules, and c represents the number of 1 displayed in both the A and B molecules.

In step (2), the functional group is an atom or atomic group that determines the chemical properties of the compound molecule. The same functional group leads to the same or similar chemical reaction, regardless of the size of the molecule to which it belongs. The SMARTS representation of the full amount of functional groups is crawled from a Daylight chemical information system, and the functional groups are sorted by the number of atoms contained in the functional group to find out which functional group each atom in the molecule belongs to. When an atom belongs to a plurality of functional groups, a functional group having a larger number of atoms is preferentially matched as the functional group corresponding to the atom.

In step (3), modeling the molecular graph with heterogeneous graph is beneficial to characterize the different attributes of each node and edge.

The specific process of step (4) is:

taking the heterogeneous graph with initialized node features and functional group features as an input of the structure-aware molecular encoder, transferring information by the relational graph convolutional network (RGCN) in the structure-aware molecular encoder through calculating and aggregating information for different types of edges, and integrating the information aggregated by different edges for different types of nodes;

after obtaining the feature representation of each atom and the functional group that the atom belongs to, then aggregating the features of the nodes and the functional groups to obtain the structure-aware feature representation of the molecule.

A formula for the information transfer of the relational graph convolutional network (RGCN) is as follows:

$${h}_{i}^{l + 1} = σ ( {\sum }_{r ∈ R} {\sum }_{j ∈ {N}_{i}^{r}} \frac{1}{{c}_{i , r}} {W}_{r}^{l} {h}_{j}^{l} + {W}_{0}^{l} {h}_{i}^{l} )$$

wherein, R is a set of all edges, Nᵢ^{r} is all neighbor nodes which are adjacent to the node i and are of edge type r, c_{i,r} is a parameter that can be learned, Wᵣ^{l} is a weight matrix of the current layer l, hᵢ^{l} is a feature vector of the current layer l to the current node i; the feature of each neighbor node is multiplied by a weight corresponding to the edge type, and then is multiplied by a learnable parameter, and then summed, and finally, the information transferred by a self-loop edge is added and the activation function σ is passed, which is used as an output of the layer and an input of a next layer.

In step (5), when selecting the positive and negative samples, one molecule of which similarity with a target molecule is greater than a certain threshold is selected as the positive sample, K molecules of which each similarity is less than a certain threshold are selected as the negative samples; a feature representation corresponding to the target molecule is denoted as q, a feature representation of the positive sample is denoted as k₀, and the feature representations of K negative samples are denoted as k₁, . . . , k_{K}.

After obtaining the feature representations of each target molecule and the positive and negative samples thereof, a loss is calculated by using a loss function, and the parameters of the structure-aware molecular encoder are updated through a back-propagation algorithm, which causes the model to recognize the target molecule and the positive samples as similar instances and distinguish the target molecule and the positive samples from dissimilar samples.

The loss function described is InfoNCE, and the formula is as follows:

$$L = - log \frac{exp ( {q}^{T} {k}_{0} / τ )}{{\sum }_{i = 0}^{K} exp ( {q}^{T} {k}_{i} / τ )}$$

wherein, τ is a hyperparameter, the loss function causes the model to identify the target molecule q and positive samples k₀ as similar instances, and to distinguish q from dissimilar instances k₁, . . . , k_{K}.

The specific process of step (6) is:

training the structure-aware molecular encoder on the large-sample molecular data set through the contrastive learning method described in step (5); then inputting molecular data in a small-sample data set into the structure-aware molecular encoder, and then using a linear classifier to classify the molecular representations output by the encoder, and predicting the molecular attributes.

Compared with the prior art, the present invention has the following beneficial effects:

1. Different from the existing supervised pre-training method, the present invention uses the self-supervised contrastive learning method to train the structure-aware molecular encoder. Supervised learning has the problem of insufficient labeled data, and the model obtained through label training often only involve specific knowledge, which is far less rich than the structural information of the data itself. Therefore, using the self-supervised contrastive learning method to construct labels based on the structure or characteristics of the molecular graph data itself for molecular graph representation learning is helpful to capture richer molecular structural information, and it is easier to obtain discriminative high-level features.

2. In the present invention, modeling the molecular graph with the heterogeneous graph is beneficial to characterize the different attributes of each atom and bond.

3. Different from the existing molecular graph representation learning methods lacking prior knowledge in the field of chemistry, the present invention proposes to use the structure-aware graph neural network to learn the molecular representation, and directly encode the functional group information that plays a decisive role in molecular properties into the feature representation of the graph.

### BRIEF DESCRIPTION OF THE DRAWINGS

In order to illustrate the embodiments of the present invention or the technical solutions in the prior art more clearly, the following briefly introduces the accompanying drawings used in the description of the embodiments or the prior art. Obviously, the drawings in the following description are only some embodiments of the present invention. For those of ordinary skill in the art, other drawings can also be obtained from these drawings without creative efforts.

FIG. 1 is a schematic flowchart of a molecular graph representation learning method based on contrastive learning provided by an embodiment of the present invention;

FIG. 2 is a schematic structural diagram of a structure-aware molecular encoder provided by an embodiment of the present invention.

### DETAILED DESCRIPTION OF THE EMBODIMENTS

The present invention will be further described in detail below with reference to the accompanying drawings and embodiments. It should be pointed out that the following embodiments are intended to facilitate the understanding of the present invention, but do not have any limiting effect on it.

The molecular graph representation learning method based on contrastive learning provided by the present invention can be used in application scenarios such as a chemical molecule attribute prediction, virtual screening, etc., and selects positive and negative samples based on similarities of molecular fingerprints, and compares them with molecular data in a feature space, and directly encodes the knowledge of functional groups in the chemical field into the representations of the molecules to obtain a molecular graph representation with chemical field knowledge and distinctiveness. The present invention solves the problem of insufficient labeling data in supervised learning, and makes full use of the structure or characteristics of the molecular map data itself to construct labels.

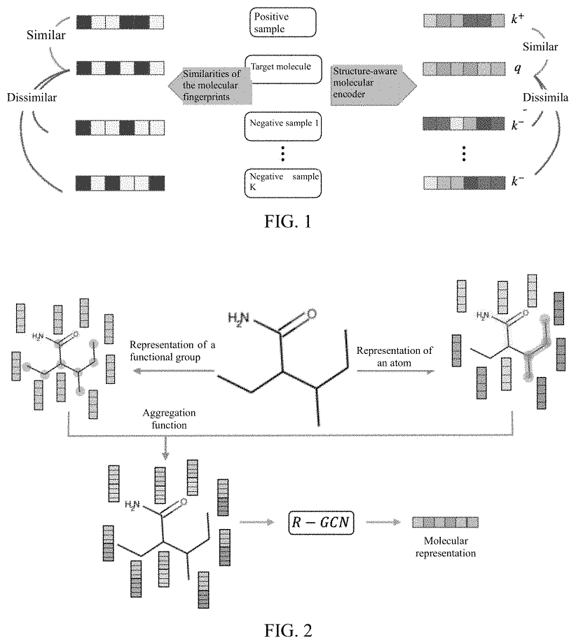

As shown in FIG. 1, a molecular graph representation learning method based on contrastive learning, wherein, the method comprises the following steps:

firstly, transforming the SMILES representation of the molecules into the molecular fingerprint by Rdkit which is a powerful tool for cheminformatics. For each molecule, after calculating the fingerprint similarities between it and all other molecules using a Tanimoto coefficient, selecting one molecule of which the similarity with the molecule is greater than a certain threshold as a positive sample, and selecting K molecules of which the similarities are less than a certain threshold as negative samples.

Modeling the target molecule and its corresponding positive and negative samples by using a heterogeneous graph, which aims to characterize the different attributes of each node and edge. Inputting the sample data of the molecules into a structure-aware molecular encoder shown in FIG. 2, and obtaining the feature representations corresponding to the target sample and the positive and negative samples. Denoting a feature representation corresponding to the target molecule as q, denoting a feature representation of the positive sample as k₀, and denoting the feature representations of K negative samples as k₁, . . . , k_{K}.

Taking InfoNCE as a loss function, the parameters of the model are updated through a back-propagation algorithm, which encourages the model to identify the target molecule and positive samples as similar instances, and at the same time distinguish them from dissimilar instances to learn discriminative structure-aware molecular feature representation.

The loss function described is InfoNCE, and the formula is as follows:

$$L = - log \frac{exp ( {q}^{T} {k}_{0} / τ )}{{\sum }_{i = 0}^{K} exp ( {q}^{T} {k}_{i} / τ )}$$

wherein, τ is a hyperparameter, the loss function causes the model to identify the target molecule q and positive samples k₀ as similar instances, and to distinguish q from dissimilar instances k₁, . . . , k_{K}.

As shown in FIG. 2, it is a schematic diagram of a structure-aware graph neural network provided by an embodiment of the present invention. Modeling the molecules by using the heterogeneous graph with initialized node features and functional group features, and characterizing the different attributes of each node and edge. Taking the heterogeneous graph as an input of the structure-aware molecular encoder, and then calculating and aggregating information for different types of edges by utilizing RGCN, and integrating the information aggregated by different edges for different types of nodes to transfer information. The RGCN takes into account the type of edge, and in order to transfer the features of the nodes in a previous layer to a next layer, the RGCN adds a special self-loop edge for each node. The specific information transfer process is as follows:

$${h}_{i}^{l + 1} = σ ( {\sum }_{r ∈ R} {\sum }_{j ∈ {N}_{i}^{r}} \frac{1}{{c}_{i , r}} {W}_{r}^{l} {h}_{j}^{l} + {W}_{0}^{l} {h}_{i}^{l} )$$

wherein, R is a set of all edges, Nᵢ^{r} is all neighbor nodes which are adjacent to the node i and are of edge type r, c_{i,r} is a parameter that can be learned, Wᵣ^{l} is a weight matrix of the current layer l, hᵢ^{l} is a feature vector of the current layer l to the current node i. The feature of each neighbor node is multiplied by a weight corresponding to the edge type, and then is multiplied by a learnable parameter, and then summed, and finally, the information transferred by a self-loop edge is added and the activation function σ is passed, which is used as an output of the layer and an input of a next layer.

After obtaining the feature representation of each atom and the functional group that the atom belongs to by the RGCN, then aggregating the features of the nodes and the functional groups by an aggregation function to obtain the structure-aware feature representation of the molecule.

The above-mentioned embodiments describe the technical solutions and beneficial effects of the present invention in detail. It should be understood that the above-mentioned embodiments are only specific embodiments of the present invention and are not intended to limit the present invention. Any modifications, additions and equivalent substitutions made within the scope of the principles of the present invention shall be included within the protection scope of the present invention.

1. A molecular graph representation learning method based on contrastive learning, wherein, the method comprises the following steps:
(1) obtaining a molecular fingerprint representation of each molecule, and calculating a similarity between each two molecular fingerprints;
(2) collecting a full amount of chemical functional group information, and matching a corresponding functional group for each atom in the molecule; wherein, when an atom belongs to a plurality of functional groups, a functional group containing a larger number of atoms is preferentially matched as the functional group corresponding to the atom;
(3) using a heterogeneous graph to model a molecular graph, wherein the heterogeneous graph is a graph containing different types of nodes and edges, different atoms correspond to different node types, and different bonds correspond to different edge types;
(4) constructing a structure-aware molecular encoder, using a relational graph convolutional network (RGCN) in the structure-aware molecular encoder to encode the representation of each atom in the molecule and the representation of the functional group to which the atom belongs, and mapping the molecule to a feature space through an aggregation function to obtain a structure-aware feature representation, wherein the specific process is as follows:
taking the heterogeneous graph with initialized node features and functional group features as an input of the structure-aware molecular encoder, transferring information by the relational graph convolutional network (RGCN) in the structure-aware molecular encoder through calculating and aggregating information for different types of edges, and integrating the information aggregated by different edges for different types of nodes; after obtaining the feature representation of each atom and the functional group that the atom belongs to, then aggregating the features of the nodes and the functional groups to obtain the structure-aware feature representation of the molecule;
wherein, a formula for the information transfer of the relational graph convolutional network (RGCN) is as follows:
$${h}_{i}^{l + 1} = σ ( {\sum }_{r ∈ R} {\sum }_{j ∈ {N}_{i}^{r}} \frac{1}{{c}_{i , r}} {W}_{r}^{l} {h}_{j}^{l} + {W}_{0}^{l} {h}_{i}^{l} )$$
wherein, R is a set of all edges, Nᵢ^{r} is all neighbor nodes which are adjacent to the node i and are of edge type r, c_{i,r} is a parameter that can be learned, Wᵣ^{l} is a weight matrix of the current layer l, hᵢ^{l} is a feature vector of the current layer l to the current node i; the feature of each neighbor node is multiplied by a weight corresponding to the edge type, and then is multiplied by a learnable parameter, and then summed, and finally, the information transferred by a self-loop edge is added and the activation function σ is passed, which is used as an output of the layer and an input of a next layer;
(5) according to the fingerprint similarity between molecules, selecting positive and negative samples, and carrying out a comparative learning in the feature space;
(6) obtaining the structure-aware molecular encoder by using the contrastive learning method for training on a large-sample molecular dataset, and applying the structure-aware molecular encoder to a prediction task of downstream molecular attributes.

2. The molecular graph representation learning method based on contrastive learning according to claim 1, wherein in step (1), a Simplified Molecular Input Line Entry System (SMILES) representation of each molecule is transformed into the molecular fingerprint through Rdkit; the molecular fingerprint is selected from one of Morgan fingerprints, Molecular ACCess System (MACCs) fingerprint and topology fingerprint.

3. The molecular graph representation learning method based on contrastive learning according to claim 2, wherein in step (1), the similarity between two molecular fingerprints is calculated using a Tanimoto coefficient, and the formula is as follows:
$${S}_{AB} = \frac{c}{a + b - c}$$
wherein, the partial molecular structures of 166 molecules are pre-specified by MACCs fingerprints; when any molecular structure is contained, the corresponding position is recorded as 1, otherwise, it is recorded as 0; a and b respectively represent the number of 1 displayed in the A and B molecules, and c represents the number of 1 displayed in both the A and B molecules.

4. The molecular graph representation learning method based on contrastive learning according to claim 1, wherein, in step (5), when selecting the positive and negative samples, one molecule of which similarity with a target molecule is greater than a certain threshold is selected as the positive sample, K molecules of which each similarity is less than a certain threshold are selected as the negative samples; a feature representation corresponding to the target molecule is denoted as q, a feature representation of the positive sample is denoted as k₀, and the feature representations of K negative samples are denoted as k₁, . . . , k_{K}.

5. The molecular graph representation learning method based on contrastive learning according to claim 4, wherein, after obtaining the feature representations of each target molecule and the positive and negative samples thereof, a loss is calculated by using a loss function, and the parameters of the structure-aware molecular encoder are updated through a back-propagation algorithm, which causes the structure-aware molecular encoder to recognize the target molecule and the positive samples as similar instances and distinguish the target molecule and the positive samples from dissimilar samples.

6. The molecular graph representation learning method based on contrast learning according to claim 5, wherein the loss function is InfoNCE, and the formula is as follows:
$$L = - log \frac{exp ( {q}^{T} {k}_{0} / τ )}{{\sum }_{i = 0}^{K} exp ( {q}^{T} {k}_{i} / τ )}$$
wherein, τ is a hyperparameter, the loss function causes the model to identify the target molecule q and positive samples k₀ as similar instances, and to distinguish q from dissimilar instances k₁, . . . , k_{K}.

7. The molecular graph representation learning method based on contrast learning according to claim 1, wherein the specific process of step (6) is as follows:
training the structure-aware molecular encoder on the large-sample molecular data set through the contrastive learning method described in step (5); then inputting molecular data in a small-sample data set into the structure-aware molecular encoder, and then using a linear classifier to classify the molecular representations output by the encoder, and predicting the molecular attributes.
